(19) United States
(12) Patent Application Publication
(10) Pub. No.: US 2017/0001024 A1
(43) Pub. Date: Jan. 5, 2017

(54) HIGH POWER TIME VARYING MAGNETIC FIELD THERAPY

(71) Applicant: BTL HOLDINGS LIMITED, Limassol (CY)

(72) Inventor: Ondra Prouza, Ricany u Prahy (CZ)

(21) Appl. No.: 14/926,365

(22) Filed: Oct. 29, 2015

Related U.S. Application Data
(63) Continuation-in-part of application No. 14/789,156, filed on Jul. 1, 2015, now abandoned.
Application No. 14/789,156 is a continuation-in-part of application No. 14/789,658, filed on Jul. 1, 2015.

(51) Int. Cl. A61N 2/00; A61N 2/02

(52) U.S. Cl. CPC A61N 2/004; A61N 2/02

(57) ABSTRACT

In a method for stimulation and treatment, a biological structure is stimulated by a high power time-varying magnetic field. The stimulation is followed by at least a partial muscle contraction. The methods can be used e.g. in physiotherapy, urology or urogynecology.

This Application is a Continuation-in-Part of U.S. patent application Ser. No. 14/789,156 filed Jul. 1, 2015, and now pending. This Application is also a Continuation-in-Part of U.S. patent application Ser. No. 14/789,658 filed Jul. 1, 2015, and now pending. Application Ser. Nos. 14/789,156 and 14/789,658 are incorporated herein by reference.

### BACKGROUND OF THE INVENTION

Magnet therapy uses the influence of magnetic flux on biological tissue. Electric current is induced in the tissue due to voltage change which causes a polarization of the cell membrane. One of fundamental phenomenon of electric current in biological tissue is a transfer of neural excitation or muscle contraction. The intensity of the effect is dependent on the magnetic flux density, repetition rate of the pulses, pulse time duration or envelope of the stimulation signal.

Water and biological molecules are diamagnetic substances. The magnetic field is not affected by diamagnetic substances. Therefore no loss of intensity or magnetic flux density occurs when passing through the biological structure or tissue.

Magnet therapy originally used permanent magnets with a stationary magnetic field. Natural magnets were applied especially to acupuncture points, or to the location of pain. Thereafter natural magnets were replaced by synthetic magnets and electromagnets of stationary magnetic field of higher induction than permanent magnets. In the last few decades, therapeutic methods have used mainly a pulsed magnetic field.

Existing methods of magnetic therapy generally tend to be limited to the key parameters of magnetic flux density and repetition rate. High values of magnetic flux density are reached at low repetition rate or vice versa. These combinations limit the effectiveness of muscle therapy at higher repetition rates over 50 Hz. Therefore the stimulation of deep structures or stimulation by high repetition rates or the combination of both is limited. Existing designs do not provide any device and/or method for stimulating biological structure at repetition rate over 50 Hz and magnetic flux density sufficient to cause at least partial muscle contraction repetitively. Additionally existing methods do not disclose time duration of the therapy.

Existing methods are also not able to provide stimulation of biological structures by pulsed magnetic field at repetition rates which exceed the frequency resolution of the biological structure. Some systems also require making physical contact with the patient since the magnetic field is weak or the stimulation signal cannot be transferred without the electrical contact. Generally, these known methods are limited to repetition rates over 50 Hz in order to provide biological structure stimulation. Furthermore, repetition rates exceeding 100 Hz are not utilized. The therapeutic methods at higher repletion rates over 100 Hz are provided only by electrotherapeutic methods.

Presently, muscle contraction leading to strengthening, training, myorelaxation or analgesic effect at higher repetition rates over 50 Hz and at sufficient intensity stimulus may be achieved only by direct current therapy. However, direct current methods require contact with the patient and even may be invasive. These methods can result in skin irritation, painful application especially for the stimulus of higher intensity, discomfort during the treatment, lack of deep tissue stimulation by non-invasive methods, and a lack of patient compliance with a prescribed therapy due to these factors.

### SUMMARY OF THE INVENTION

In a first aspect, a method provides stimulation of biological structure using magnetic field at repetition rates exceeding 50 Hz for purpose of at least a partial muscle contraction.

The method may provide a non-invasive transfer of a stimulation signal from an applicator to biological structure to evoke the action potential of biological structure.

The method may use a peak to peak magnetic flux density on a coil surface at least 0.2 T, 0.4 T, 1.5 T, 2 T, or at least 3 T. The repetition rate may exceed 50 Hz, 80 Hz, 90 Hz, 100 Hz or 120 Hz, and up to 150 Hz, with preferable repetition rate up to 700 Hz. with initial or successive treatments lasting several seconds or longer, for example, for at least 5, 10, 30, 60, 120 or 240 seconds, or longer. The pulse width is in the range of tens to hundreds of microseconds.

In another aspect of the invention, a neuromuscular plate is stimulated causing an at least partial contraction of the muscle. The muscle is contracted at higher repetition rates and the contraction is stronger and more efficient for improving the muscle strength. The method is especially useful for deep muscles, major muscles, and for treatment of patients with high value of BMI. Deep muscle is the muscle underneath the superficial muscle. Muscle tissues may be selectively stimulated and the magnetic flux density of the stimulation may be adjusted based on patient characteristics or input. Treatment time can be shortened to a minimum due to selective stimulation of targeted muscles. Additionally, the treatment may be non-invasive or even contactless due to the high value of magnetic flux density. The patient may be treated without removing clothing, thereby reducing patient discomfort.

The target biological structure may be a joint. Due to the pulsed magnetic field, the dynamic fluid properties of synovial fluid are improved and muscle contraction is achieved, contributing to positioning of the joint by short movements of the joint compartments.

In another aspect of the invention the repetition rate may exceed the frequency resolution of the structure. The magnetic flux density of the stimulation signal may increase over time. Therefore the envelope of resulting stimulation signal is increasing and it is perceived by the stimulated biological structure as a continuous stimulation signal instead of plurality of discrete stimuli. The envelope may be preferably triangular and other shapes may be used as well. This method is effective for stimulation of denervated muscle.

In a further aspect of the invention, the method stimulates the biological structure via a magnetic stimulation signal of at least 100 Hz, where the stimulation is intended for at least partial muscle contraction. The pulsed magnetic field induces the electric current which may provide myorelaxation. The stimulation signal repetition rate may be at least 120 Hz or at least 140 Hz.

### BRIEF DESCRIPTION OF THE DRAWINGS

FIG. 1 illustrates a curve of action potential of a biological structure.

FIG. 2 illustrates a threshold value corresponding to different envelopes of the stimulation signal.

FIG. 3 illustrates a detail of a stimulation signal with increasing envelope.

### GLOSSARY

Biological structure/target biological structure includes a cell, a neuron, a nerve, a muscle fibre, a tissue, a filament.

Stimulation signal refers to a magnetic flux density inducing an electric current in the biological structure.

Active response of a biological structure includes a change in a permeability of cell membrane for ions or any other particles, generation of an action potential, at least partial muscle contraction, a change of rheological properties of synovial fluid.

Sensory intensity is the stimulation intensity when the patient feels the first perception of the induced current flow in the stimulated biological structure.

Motoric intensity is the stimulation intensity when the patient registers the first muscle contraction.

Noxious intensity is the stimulation intensity when the patient recognizes first painful stimulus.

Impulse refers to the only one biphasic magnetic stimulus.

Pulse refers to a period of stimulation signal consisting of at least one biphasic stimulus and a time duration of no stimulation, i.e. time duration between two impulses from rise edge to next rise edge.

Repetition rate refers to frequency of firing the pulses; it is derived from the time duration of a pulse.

### DETAILED DESCRIPTION OF THE INVENTION

Electric current is induced in the stimulated biological structure during pulsed magnet therapy. A distribution of magnetic field is uniform in the biological structure. Particles (e.g. atoms, ions, molecules etc.) in the biological structures are affected by the magnetic field and permeability of a cell membrane also increases.

Due to increased permeability of the cell membrane, an action potential may occur and a partial or full muscle contraction is induced. Convenient repetition rates may cause pain relief and/or myorelaxation, different repetition rate may cause stimulation of denervated muscle, and further different repetition rates may improve movability of a joint.

Advantages of the present magnet therapy include: affecting the deep structures which are problematically stimulated by superficial stimulation; non-invasive or non-contact application of magnetic flux, it may be applied even with clothes; absolute non-invasiveness of the stimulation and elimination of skin irritation in the place of magnetic field application; high rate of acceptability of the stimulation by patients; elimination of stimulation side effects; elimination necessity of applicator made of biocompatible materials; providing a clean and sterile applicator on the highest level; possibility of local or area treatment.

It is to be understood that the method is not limited to the particular applications and that the method may be practiced or carried out in various ways.

Present invention relates to methods using magnetic stimulation of magnetic flux density at least sufficient to cause active response of a biological structure at the repetition rates at least 50 Hz. The broad spectrum of application of biological structure stimulation by magnetic field is achieved due to high repetition rates and/or high value of magnetic flux density. Methods are intended especially for at least partial muscle contraction.

The present methods may be provided by the magnetic stimulation device which contains no magnetic core, however, the magnetic core may also be used. The magnetic stimulation device may be cooled by a fluid, such as air. The total power consumption may be preferable, but is not limited to values below 1.3 kW. Convenient therapeutic apparatus is described in the U.S. patent application Ser. No. 14/789,156 or U.S. patent application Ser. No. 14/789,658, incorporated herein by reference.

The applicator for magnet therapy is placed proximate to the patient's body. The magnetic flux is applied into the biological structure. The electric current is induced and stimulates the neuromuscular plate. Due to the stimulation at least a partial muscle contraction is caused.

The present method stimulates the biological structure by pulsed magnetic field. The peak to peak magnetic flux density on the coil surface is at least 0.2 T, 0.4 T, 1.5 T, 2 T, or at least 3 T and with magnetic flux density up to 7 T at repetition rates exceeding 50 Hz, 80 Hz, 90 Hz, 100 Hz, or 120 Hz with treatment/successive treatments lasting several seconds or longer, for example, for at least 5, 10, 30, 60, 120, or 240 seconds, or longer. The pulse width is in the range of tens to hundreds of microseconds.

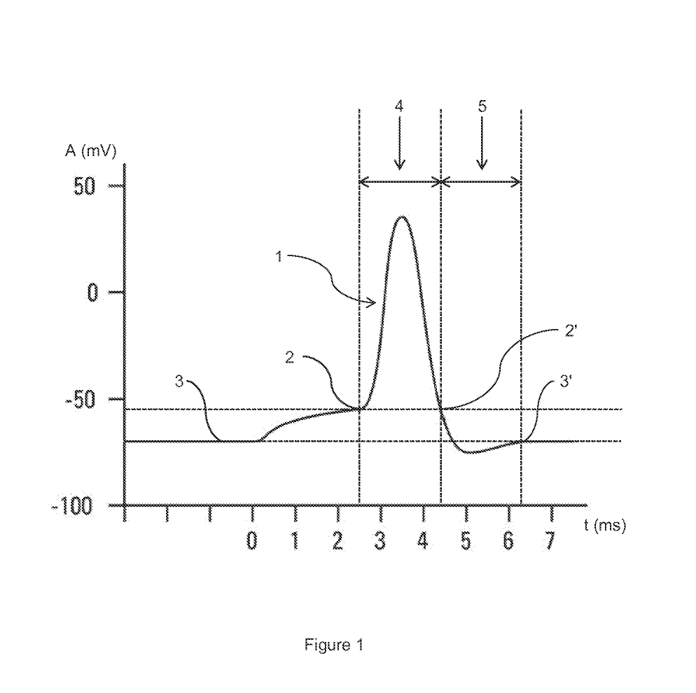

Referring to FIG. 1, the stimulation of the biological structure, a cell, is explained by a curve 1 of an action potential. The action potential of the cell rapidly increases after the stimulus (induced by pulsed magnetic field) and reaches the threshold 2—so called depolarization. After the reaching the maximal amplitude value, the membrane permeability changes and repolarization occurs. The negative value is reached in relation to resting potential 3. Afterwards the potential recharges back to the value of resting potential 3′. The time period from the threshold 2 to the return of potential to the threshold 2′ (which equals threshold value 2) is called absolute refractory period 4. The cell is not able to be stimulated any more during the absolute refractory period 4, even by very strong stimulus. The time period from the end of absolute refractory period 4 to resting potential 3′ is called relative refractory period 5. The cell is able to be stimulated only by the intensive over-threshold stimulus during the relative refractory period 5. Over-threshold stimulus is a stimulus of higher magnetic flux density than the value of threshold stimulus. The absolute refractory period 4 is the same time duration for all the cells, however, the relative refractory period 5 varies following the cell type.

The present methods may be used for muscle stimulation and exercising, e.g. for treatment of pelvic floor muscles, incontinence, etc. Incontinence is a disability of mainly older patients to restrain evacuations of urine or feces. Incontinence is currently treated by exercising pelvic floor muscles or by utilizing vaginal or rectal probes using direct current therapy, or using urologic chairs using stimulation by pulsed magnetic field. However, urologic chairs achieve low magnetic flux density at high repetition rate. Efficacy of urological chairs is low because the values of stimulation parameters, repetition rate and magnetic flux density, are insufficient. Therefore the therapy is relatively time consuming and uncomfortable for the patient.

Another field of application may be treatment of erectile dysfunction. A synergic effect may be also be myorelaxation.

The present methods may be used for various stimulation of any other muscle or muscle group or any other biological structure, especially for the deep muscles, e.g. psoas major muscle, and the diaphragm. The method may stimulate other biological structures, e.g. selective stimulation of the particular muscle groups for improving their functionality or for generating a contraction pattern from the muscle group for improving the efficiency of the movement or generating the muscle memory.

According to another aspect of this invention the method may be used for magnetic field stimulation by pulsed magnetic field at repetition rates above 50 Hz, 100 Hz, 150 Hz, or 200 Hz are favourable mainly for treatment of denervated muscle. Denervated muscle lacks the ability of conscious contraction due to a lesion or nerve degradation caused by e.g. polio or trauma, so that the signals from the central neural system are not received. The muscle loses the ability of contraction and flexibility and it atrophies. Effects of muscle atrophy are visible just after three weeks of inactivity.

The stimulation of denervated muscle is based on the adaptability of health motor unit for the raising magnetic flux density. Denervated muscle lacks an ability of adaptation to raise induced electric stimulus as in a normal healthy muscle. Hence the denervated muscle is stimulated by low magnetic flux density.

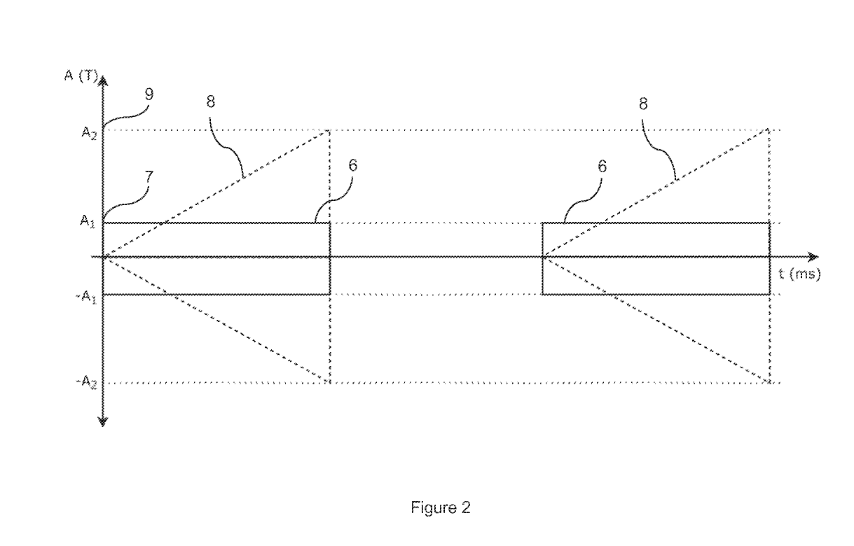

FIG. 2 illustrates the different shapes of the envelope of the stimulation signal and corresponding different threshold values of a healthy muscle.

When the healthy muscle is stimulated by a rectangular envelope 6 of stimulation signal the muscle contraction occurs at magnetic flux density A₁ 7. When the healthy muscle is stimulated by increasing envelope 8 of stimulation signal the muscle contraction occurs at magnetic flux density value A₂ 9. However, when the denervated muscle is stimulated by increasing envelope 8 of stimulation signal the denervated muscle contraction occurs at magnetic flux densities below A₂ 9. Magnetic flux density value A₂ 9 is multiplication of magnetic flux density value A₁ 7, wherein the multiplication coefficient is positive number greater than 1.

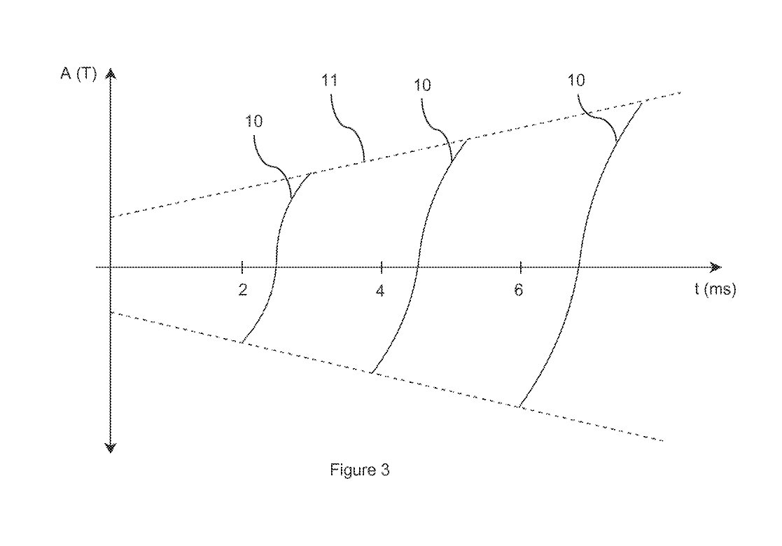

FIG. 3 describes generation of various types of envelopes. The stimulation signal includes impulses 10 with increasing magnetic flux density.

Therefore the envelope 11 is increasing and the envelope is modulated in a magnetic flux density domain. The upper value of stimulation of denervated muscle is limited by the time duration of the absolute refractory period. The method of denervated muscle stimulation by magnetic field utilizes the properties of stimulation ability of biological structure. The principle is the stimulation by the signal of sufficient repetition rate and magnetic flux density in a manner that the envelope 11 of the stimulation signal is increasing magnetic flux density and the time duration of the envelope 11 lasts at least 6 ms, 10 ms or 20 ms. The effect is achieved by stimulation of repetition rate at least 100 Hz, 150 Hz, 200 Hz, or 250 Hz.

The stimulation signal may consist of several impulses 10 called burst and a time period with no stimulation. The number of pulses in one burst varies in range of at least 2 pulses to thousands of pulses. The envelope may consist of stimulation signals with various burst frequencies, e.g. 5, 10, 20, 50, or more Hz.

The above defined method may be used for therapy, mainly for treatment of denervated muscle. Further the envelope may consist of several periods of stimulation signal of different repetition rates. Therefore the modulation of the signal is in repetition rate domain. In yet another approach the envelope may be modulated by combination of repetition rate domain and magnetic flux density domain.

The stimulation results in an at least partial contraction of denervated muscle and the contraction of healthy muscle is eliminated or minimized.

Magnetic stimulation at high repetition rates according to proposed invention is further applicable for the effect of myorelaxation. The repetition rate of at least 100 Hz, 110 Hz, 120 Hz, or 130 Hz may be used for the purpose of the muscle stimulation.

One of the myorelaxation approaches is muscle adaptation to high repetition rate of stimuli. The magnetic flux density is sufficient to induce current in the biological structure causing motoric intensity or over-motoric intensity at the beginning of the application and it becomes below-motoric intensity after several minutes at constant magnetic flux density since the muscle can accommodate for the stimulus of constant repetition rate. The impulse time duration is in at least tens of μs, more preferably at least 250 μs or 500 μs. The time duration of a pulse is at least 1 ms, 2.5 ms or 5.5 ms. A repetition rate of stimulation signal around 180 Hz is highly effective. A temporary reflex adjustment of hypertonic muscles or muscle groups is achieved by the application of magnetic stimulation signal thereafter myorelaxation is provided.

Further application of magnet therapeutic method using sufficient magnetic flux density at high repetition rates over 100 Hz is focused especially on spastic muscle and its trigger point. The method proposes stimulation of muscle hypertonia of the overloaded muscle fibres to relieve a local spasm (the hypertonia in stimulated muscle fibre is relieved). The method affects the muscle insertion as well. The method may even affect the whole muscle group for specific movement.

The stimulation by pulsed magnetic field is divided into two separate periods. The neuromuscular plate is stimulated by pulsed magnetic field during the first period. The magnetic flux density is sufficient to induce at least motoric intensity of electric current to cause at least partial muscle contraction in the stimulated biological structure. The muscle is activated by isometric contraction and sedation follows. The most reactive fibres are selectively inhibited. During the second period the repetition rate is increased to at least 100 Hz, 150 Hz, or 200 Hz. The muscle is relaxed due to high repletion rate. The method is used for high-quality relaxation of at least one muscle fibre.

The method may be used also in sport medicine for stretching of athletes before a performance and muscle relaxation after the performance, thereafter it significantly contributes to muscle regeneration. Further applications are treating for muscle imbalance or muscle relaxation caused by overload, pain relief or elimination and preparation the muscle for physical activity.

The present method may also be used for functional joint blockade treatment. A joint may include muscular structure providing the movement of the joint, joint itself including synovial fluid. Functional joint blockade may be caused by spastic muscles in the vicinity of the joint whose secondary effect is pain. The most common functional joint blockades are in the backbone. These can cause vertebrogenic problems, headache and backbone pain, migraine, perfusion ailment resulting to dizziness, backbone sharp pain directing to limbs, visual insufficiency, tinnitus, toothache or earache etc. The scoliosis, sacral pain (even after fractures), weakness of pelvic floor muscles and incontinence, urine retention or constipation or functional sterility may also be treated by treatment of functional joint blockade.

For the purpose of functional blockade treatment and joint movability improvement the preferred repetition rates may be at least 50 Hz, 60 Hz, 70 Hz or 100 Hz, magnetic flux density at least 0.2 T, 0.4 T, 0.5 T, or at least 1 T and up to 7 T.

Formerly the functional joint blockades were treated by manual positioning, traction, mobilization of the soft tissues, reflex therapy or even pharmacologically. The present method achieves at least partial muscle contraction in the vicinity of the joint, e.g. backbone, by stimulation of neuromuscular plate by pulsed magnetic field at low repetition rate and high magnetic flux density providing at least partial muscle contraction. The joint contact surfaces are moved and the joint is moved due to at least partial muscle contractions of muscles in the vicinity of the joint, e.g. the muscles in the vicinity of the backbone are represented by rotators which causes local microrotations of the backbone. The functional joint blockade is unblocked via muscle mobilization by mechanical approach.

Still another approach of the present method is affecting the rheological properties of synovial fluid by pulsed magnetic field. The total effect of this method may be synergic. Although the method is explained on example of backbone functional joint blockade, the application of the method is not limited to the backbone. A person skilled in anatomy or physical therapy is able to apply the method for any other joint provided by sufficient amount of neuromuscular structures in vicinity. The present method may be preferably used in combination with analgesic methods. It is very convenient to use the present method in combination with method providing myorelaxation since the functional joint blockade is caused by spastic biological structures in vicinity.

All the described methods of stimulation provide trophotropic, anti-oedematous or placebo effect which contributes to the patient's health state and comfort. Local metabolism may be increased as well.

The values of magnetic flux density and repetition rate are cited in several preferred applications since the perception of the stimulation is subjective. Nevertheless the magnetic flux density and repetition rates are not limited by the recited values. A person skilled in physical therapy is able to repeat and apply the therapy methods adjusting the magnetic flux density or repetition rate following the patient's needs.

A person skilled in the physical therapy is able to use various envelopes of the stimulation signal and waveform, e.g. pulse, sinusoidal, rectangular, square, triangular, saw-tooth, trapezoidal, exponential etc. for the purpose of muscle stimulation. The invention is not limited to recited shapes of stimulation signals.

Stimulation signal of biological structure by pulsed magnetic field following the recited methods may be but not limited to continuous, pulsed, randomized, burst. The pulse may be but not limited to symmetric, asymmetric, most preferably biphasic. As used here, proximate to includes in actual contact with the skin of the patient.

Thus, novel systems and methods have been described. Various changes and substitutions may of course be made without departing from the spirit and scope of the invention. The invention, therefore, should not be limited, except by the following claims and their equivalents.

### LIST OF REFERENCE NUMBERS

1 curve of action potential
2 threshold potential
2′ threshold potential after depolarization
3 resting potential
3′ resting potential after action potential
4 absolute refractory period
5 relative refractory period
6 rectangular envelope
7 magnetic flux density A1
8 increasing envelope
9 magnetic flux density A2
10 impulse
11 envelope

1-30. (canceled)

31. A method for a stimulation of a biological structure including:
a. placing a magnetic applicator proximate to at least a part of the patient's body;
b. transferring a magnetic stimulation signal from the magnetic applicator to a target biological structure;
c. generating an active response of the target biological structure wherein a repetition rate is at least 50 Hz, and a magnetic flux density is at least 0.5 T to cause (weakened) muscle exercising.

32. The method of claim 31, wherein the muscle is a deep muscle.

33. The method of claim 31, wherein the muscle is non-denervated.

34. The method of claim 31, wherein the stimulation causes at least partial muscle contraction of a muscle and/or change of rheological properties of synovial fluid for improving the movability of a joint.

35. The method of claim 31, wherein magnetic flux density is at least 1 T.

36. The method of claim 31, wherein the magnetic flux density is at least 2 T.

37. The method of claim 31, wherein the magnetic flux density is at least 3 T.

38. The method of claim 31, wherein the repetition rate is at least 70 Hz.

39. The method of claim 31, wherein the repetition rate is at least 90 Hz.

40. The method of claim 31, wherein the repetition rate is at least 110 Hz.

41. The method of claim 31, wherein the treatment lasts at least 5 seconds.

42. A method for a stimulation of a biological structure including:
a. placing a magnetic applicator proximate to at least a part of the patient's body;
b. transferring a modulated magnetic stimulation signal to a target biological structure;
c. generating an action potential in the target biological structure wherein a repetition rate is at least 50 Hz.

43. The method of claim 42, wherein the stimulation is followed by at least partial contraction of a muscle.

44. A method of claim 42, wherein the repetition rate is at least 120 Hz.

45. A method of claim 42, wherein the repetition rate is at least 150 Hz.

46. A method of claim 42, wherein the repetition rate is at least 200 Hz.

47. A method of claim 42, wherein a modulation is in repetition rate domain and/or magnetic flux density domain and/or combination of both.

48. The method of claim 44, wherein an envelope is generated.

49. The method of claim 48, wherein the envelope may be in different shapes.

50. A method of claim 48, wherein a magnetic flux density in the biological structure increases over a time interval.

51. A method of claim 50, wherein the method causes the stimulation of a denervated muscle while the contraction of health muscle is eliminated.

52. A method for a stimulation of a biological structure including:
a. placing a magnetic applicator proximate to at least a part of the patient's body;
b. transferring a magnetic stimulation signal to a target biological structure;
c. generating an action potential in the target biological structure wherein a repetition rate is at least 100 Hz; and
wherein the stimulation causes myorelaxation effect.

53. The method of claim 52, wherein the stimulation is followed by at least partial contraction of a muscle.

54. The method of claim 52, wherein the repetition rate is at least 110 Hz.

55. The method of claim 52, wherein the repetition rate is at least 120 Hz.

56. The method of claim 52, wherein the repetition rate is at least 130 Hz

57. The method of claim 52, wherein at least an isometric contraction occurs during the stimulation.

58. The method of claim 52, wherein the adaptation for stimulation occurs.

59. The method of claim 52, wherein the method causes pain relief.

60. The method of claim 52, wherein the treatment lasts at least 5 seconds.
